(19) United States
(12) Patent Application Publication
(10) Pub. No.: US 2024/0316301 A1
(43) Pub. Date: Sep. 26, 2024

(54) MECHANICAL VENTILATOR CLOSED LOOP CONTROL SYSTEM, METHODS, AND APPARATUS

(71) Applicant: Nihon Kohden OrangeMed, LLC, Santa Ana, CA (US)

(72) Inventors: Hong-Lin Du, Irvine, CA (US); Germano Forti, JR., Irvine, CA (US); Yufeng Li, Lake Forest, CA (US); Marcelo do Amaral Beraldo, Irvine, CA (US)

(21) Appl. No.: 18/189,655

(22) Filed: Mar. 24, 2023

(51) Int. Cl. A61M 16/00; G16H 40/63

(52) U.S. Cl. CPC A61M 16/024; G16H 40/63; A61M 2016/0027; A61M 2016/1025; A61M 2205/3303; A61M 2205/52; A61M 2230/04; A61M 2230/432

(57) ABSTRACT

A mechanical ventilator closed loop control system, methods, and apparatus are disclosed. In an example, a mechanical ventilator performs a respiratory treatment for a patient according to respiratory treatment settings, which include at least one ventilation mode. During the treatment, the mechanical ventilator receives and/or determines physiological parameter values from one or more physiological sensors. The physiological parameters may be patient neurological parameters, metabolic parameters, circulatory parameters, and/or respiratory parameters. The mechanical ventilator uses one or more closed loop control algorithms and the physiological parameter values to determine at least some of the respiratory treatment settings and/or the ventilation mode is to be adjusted. After making the determination, the mechanical ventilator adjusts the respiratory treatment so that the treatment reflects a current health status of the patient.

### BACKGROUND

Mechanical ventilators provide ventilation by moving breathable air into and out of the lungs of patients who are physically unable to breathe sufficiently on their own. These ventilators help keep patient airways open and deliver oxygen and remove carbon dioxide. These ventilators also provide air pressure that keeps a patient's lungs from collapsing. Mechanical ventilators may be used to treat respiratory insufficiency and sleep apnea in addition to providing post-anesthesia recovery. Mechanical ventilators are also used during surgery and other instances where a patient may be unconscious for long durations of time.

Current mechanical ventilators have numerous ventilator settings that enable a clinician to optimize air delivery to a patient. Some of the ventilator settings include tidal volume, pressure control, pressure support, positive end expiratory pressure (“PEEP”), continuous positive airway pressure (“CPAP”), respiratory rate, patient spontaneous respiratory rate, patient spontaneous minute ventilation volume, fraction of inspired oxygen (“F_{I}O₂”), inspiratory time, and pressure/flow triggers. Managing the ventilator settings in response to each patient's condition and the dynamic health changes of each patient is a complex and time-consuming task.

More recently, some mechanical ventilators have closed loop control that adjusts or sets at least some ventilator settings (within designated margins) based on one or more input parameters. The input parameters are sensed by the mechanical ventilator and include, for example, an actual spontaneous respiratory rate that is compared to a target spontaneous respiratory rate, an actual minute volume that is compared to a target minute volume, or an end tidal CO₂ level that is compared to a target end tidal CO₂ level. The mechanical ventilators adjust specified settings such that the actual values from the measured input parameters approximate the target values.

A known issue with mechanical ventilators is that a patient's respiratory condition cannot be fully assessed using measurements from a mechanical ventilator alone. For instance, patient neurological parameters, metabolic parameters, and/or circulatory parameters in addition to known respiratory parameters provide a more comprehensive patient assessment. However, known mechanical ventilators do not have access to these other parameters. Additionally, known mechanical ventilators are not configured to input these other parameters and determine needed setting adjustments.

A need accordingly exists for a mechanical ventilator that adjusts one or more settings based on a closed loop control that uses patient neurological parameters, metabolic parameters, and/or circulatory parameters as inputs.

### SUMMARY

Example systems, methods, and apparatus are disclosed herein that provide closed loop control for a mechanical ventilator. The systems, methods, and apparatus overcome at least some of the issues discussed above by being configured to use values of physiological parameters including, for example, neurological parameters, metabolic parameters, circulatory parameters, and/or respiratory parameters to automatically change an operating mode and/or setting of a mechanical ventilator. The systems, methods, and apparatus may include one or more physiological sensors and/or patient monitors to obtain the neurological parameters, metabolic parameters, circulatory parameters, and/or respiratory parameters. The physiological sensors and/or patient monitors may be integrated with a mechanical ventilator, directly communicatively coupled to a mechanical ventilator, and/or indirectly communicatively coupled to a mechanical ventilator. In some embodiments, the systems, methods, and apparatus additionally or alternatively receive the neurological parameters, metabolic parameters, circulatory parameters, and/or respiratory parameters from a patient's electronic medical record (“EMR”) or other connected health database.

As discussed herein, the systems, methods, and apparatus are configured to use the physiological parameters to change an operating mode of a mechanical ventilator. The operating mode may include, for instance, a mandatory breath mode, a spontaneous breath mode, and/or a mixed mode. Additionally or alternatively, the systems, methods, and apparatus are configured to change one or more ventilator settings based at least in part on physiological parameters. Examples of ventilator settings that may be automatically adjusted include performing (or refraining from performing) a spontaneous breath trial, a P0.1 measurement using a pressure sensor of the mechanical ventilator, a negative inspiratory force (“NIF”) measurement, and/or an inspiratory trigger function with a sensitivity so sensitive that the processor attempts to induce a patient spontaneous trigger effort. Further, in some embodiments, the systems, methods, and apparatus are configured to adjust a positive end expiratory pressure (“PEEP”) ventilator setting and/or a fraction of inspired oxygen (“F_{I}O₂”) ventilator setting based on the received/measured physiological parameters.

The example systems, methods, and apparatus enable mechanical ventilators to be more responsive to actual patient conditions without constant adjustments being needed by clinicians. In addition, the systems, methods, and apparatus can be configured for mechanical ventilators that are used in healthcare facilities or at homes, where clinicians may or may not be present to adjust modes and settings. The example systems, methods, and apparatus disclosed herein accordingly improve outcomes for patients that require breathing assistance.

In an aspect of the present disclosure, any of the structure and functionality disclosed in connection with FIGS. 1 to 15 may be combined with any of the other structure and functionality disclosed in connection with FIGS. 1 to 15.

In light of the present disclosure and the above aspects, it is therefore an advantage of the present disclosure to provide a mechanical ventilator that uses closed loop control to adjust a ventilation treatment using patient physiological data including neurological parameters, metabolic parameters, circulatory parameters, and/or respiratory parameters.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1A shows a diagram of a ventilator system including a mechanical ventilator, according to an example embodiment of the present disclosure.

FIG. 1B shows a diagram of a ventilator system including a mechanical ventilator and a separate computing device, according to an example embodiment of the present disclosure.

FIG. 2 shows a diagram of the ventilator system of FIGS. 1A or 1B with a dual limb breathing circuit and an endotracheal tube, according to an example embodiment of the present disclosure.

FIG. 3 shows a diagram of the ventilator system of FIGS. 1A or 1B with a dual limb breathing circuit and a non-invasive ventilation mask, according to an example embodiment of the present disclosure.

FIG. 4 is a diagram of the example mechanical ventilator of FIGS. 1A or 1B, according to an example embodiment of the present disclosure.

FIG. 5 shows a diagram of example ventilator settings for the mechanical ventilator of FIGS. 1A or 1B, according to an example embodiment of the present disclosure.

FIGS. 6A and 6B show diagrams of control algorithms that specify certain ventilator settings based on a corresponding ventilation modes, according to an example embodiment of the present disclosure.

FIG. 7 shows a diagram of the ventilator system of FIGS. 1A or 1B with a patient monitor, according to an example embodiment of the present disclosure.

FIG. 8 shows a diagram of the ventilator system of FIG. 7 with the patient monitor connected to a network, according to an example embodiment of the present disclosure.

FIG. 9 shows a diagram of the ventilator system of FIG. 7 with the mechanical ventilator connected to a server for receiving at least some physiological parameter values, according to an example embodiment of the present disclosure.

FIG. 10 shows a flow diagram illustrating an example procedure for closed loop control of the mechanical ventilator of FIGS. 1 to 9, according to an example embodiment of the present disclosure.

FIGS. 11 to 15 show diagrams that are illustrative of at least some control algorithms used by the mechanical ventilator of FIGS. 1 to 9 to provide closed loop feedback control using physiological parameter values, according to example embodiments of the present disclosure.

### DETAILED DESCRIPTION

Methods, systems, and apparatus are disclosed for closed loop control for a mechanical ventilator. The closed loop control adjusts one or more ventilator settings and/or modes based on physiological parameters that are indicative of a patient health condition. The methods, systems, and apparatus may be used for any type of mechanical ventilator including home-based ventilators, intensive care ventilators, portable/ambulatory ventilators, and/or clinic-based ventilators. The closed loop control uses patient physiological parameter values from one or more sensors and/or patient data from a patient's medical records.

Known mechanical ventilators do not have access to comprehensive physiological parameters of patients. The known mechanical ventilators instead have closed loop control that adjusts or sets at least some ventilator settings based on internal respiratory input parameters. The input parameters are sensed by flow, pressure, and/or air sensors within the known mechanical ventilators and include, for example, an actual spontaneous respiratory rate, an actual minute volume that is compared to a target minute volume, a SpO2 level, and an end tidal CO₂ level. The input parameters are compared to target parameter values. The known mechanical ventilators make adjustments to one or more settings to cause the actual input parameter values to return to the target parameter values.

The methods, systems, and apparatus overcome limitations of known mechanical ventilators by receiving and using comprehensive patient physiological parameters to adjust modes and/or settings according to one or more control algorithms. As disclosed herein, the control algorithms relate specified physiological parameter values to one or more ventilation modes and/or settings. The control algorithms may be located within a memory device and executed on a mechanical ventilator to provide closed loop control. In alternative embodiments, the control algorithms may be executed by a cloud-based computing system or bedside patient monitor that provides remote operating instructions to the mechanical ventilator. Further, the control algorithms may be executed in separate computing device that is communicatively coupled to a mechanical ventilator.

Reference is made herein to input parameters, physiological parameters, and ventilator settings. As discussed herein, input parameters refer to respiratory-specific parameters that may be measured or determined from pressure sensors, flow rate sensors, and/or air sensors of a mechanical ventilator. The input parameters may include, for instance, a respiratory minute volume, a spontaneous respiratory rate, a pulse oximeter measurement such as Saturation of Peripheral Oxygen (“SpO₂”), and an end tidal measurement (usually of CO₂).

Ventilator settings specify how air is to be delivered to a patient. The settings may be specified individually. Alternatively, one or more settings may be set on the mechanical ventilator based on a specified target input parameter value. For instance, a clinician may specify a target respiratory minute volume. The mechanical ventilator uses a lookup table or other data structure that defines which value of settings should be selected to achieve the target respiratory minute volume. After a respiratory treatment has started, the mechanical ventilator compares the measured or calculated respiratory minute volume to the target respiratory minute volume to determine when adjustments to settings are needed such that subsequent measured respiratory minute volumes match or approximate the target respiratory minute volumes. Examples of ventilator settings include tidal volume, pressure control, pressure support, PEEP, CPAP, respiratory rate, patient spontaneous respiratory rate, patient spontaneous minute ventilation volume, F_{I}O₂”, inspiratory time, and pressure/flow triggers.

Physiological parameters specify information indicative of a patient condition. The physiological parameters may be measured by one or more sensors of a mechanical ventilator and/or one or more sensors or patient monitors that are communicatively coupled to a mechanical ventilator. The physiological parameters may also be obtained from one or more databases or serves, such as a patient's EMR. As disclosed herein, physiological parameters can include neurological parameters, metabolic parameters, and/or circulatory parameters. The physiological parameters may also include respiratory input parameters measured or determined by the mechanical ventilator.

### Ventilation System Embodiments

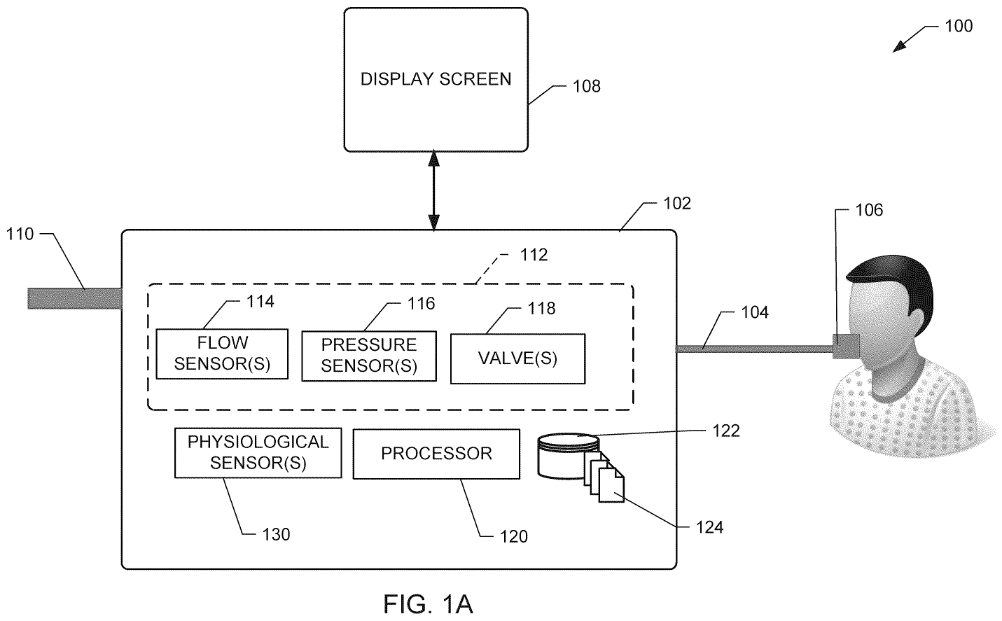

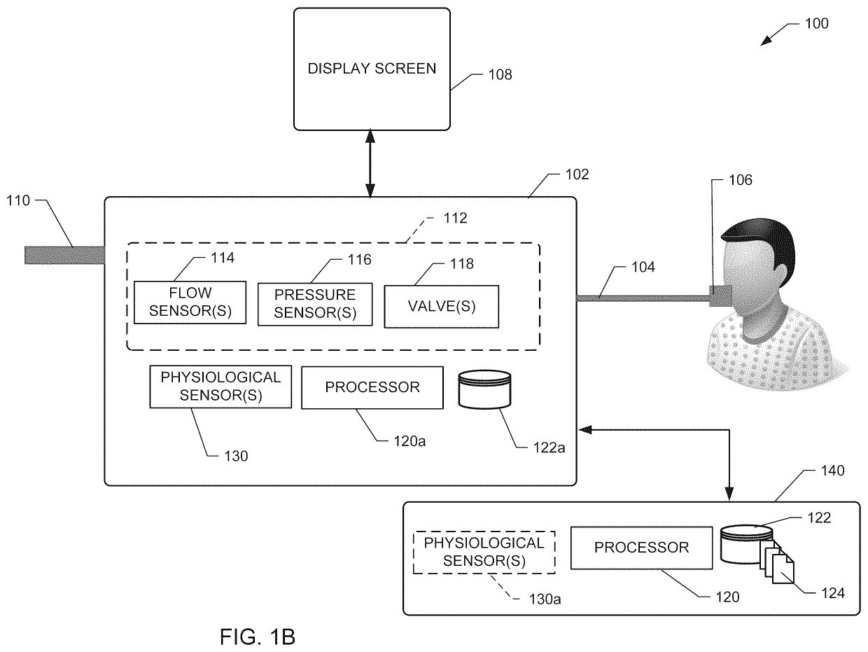

FIG. 1A shows a diagram of a ventilator system 100, according to an example embodiment of the present disclosure. The ventilator system 100 includes a mechanical ventilator 102 that is fluidly coupled to a patient via tubing 104 and a patient coupling 106. The tubing 104 may include a single limb breathing circuit, a dual limb breathing circuit, or a coax breathing circuit. In some embodiments, the tubing 104 (or an inspiratory limb of the tubing 104) may include a heated wire breathing circuit, which may include a heat moisture exchanger, a heater wire, a temperature probe, and a humidifier. The patient coupling 106 may include a nasal cannula, a non-invasive ventilation mask, an endotracheal tube, or a tracheostomy tube.

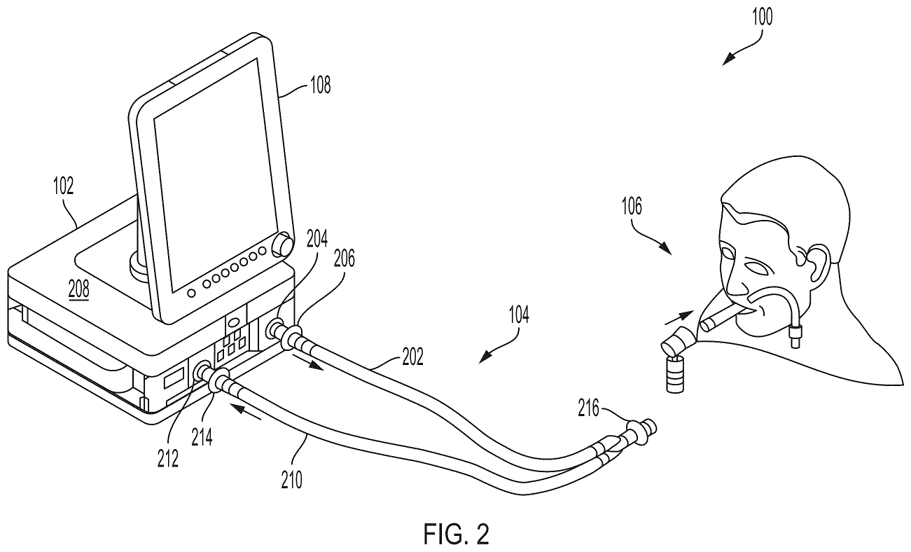

FIG. 2 shows a diagram of the ventilator system 100 of FIGS. 1A or 1B with the tubing 104 including a dual limb breathing circuit and the patient coupling 106 including an endotracheal tube, according to an example embodiment of the present disclosure. The dual limb breathing circuit includes an inspiratory limb 202 that is connected to an inspiratory port 204 via an inspiratory bio-filter 206. The inspiratory port 204 is connected to or integrally formed with a housing 208 of the mechanical ventilator 102. Air is provided to the patient for inhalation from the mechanical ventilator 102 via the inspiratory limb 202.

The dual limb breathing circuit also includes an expiratory limb 210 that is connected to an expiratory port 212 via an expiratory bio-filter 214. Air is exhaled from the patient and received in the mechanical ventilator 102 via the expiratory limb 210. Ends of the inspiratory limb 202 and the expiratory limb 210 are coupled to a heat moisture exchanger 216, which is coupled to the patient coupling 106. The heat moisture exchanger 216 may be connected to a filter or nebulizer, which is connected to an endotracheal tube.

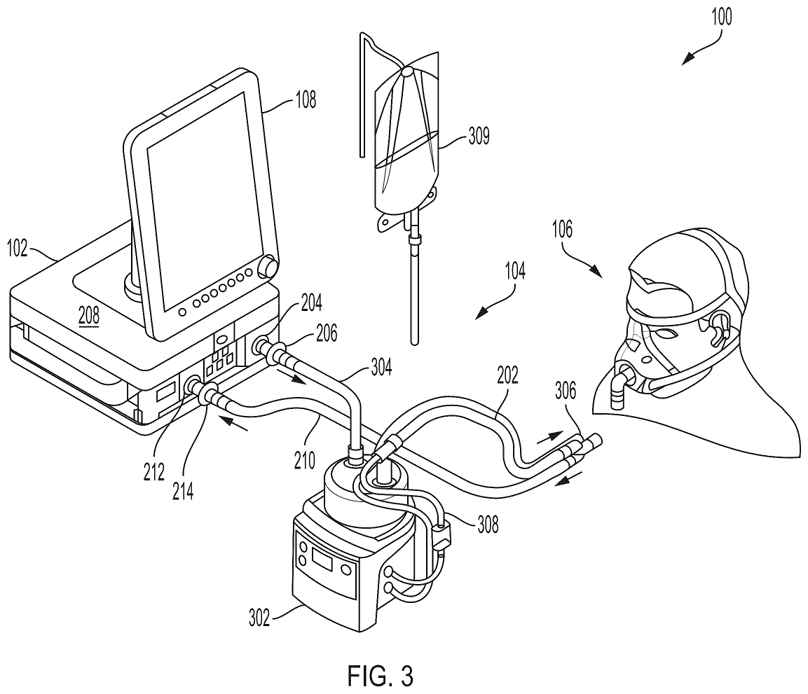

FIG. 3 shows a diagram of the ventilator system 100 of FIGS. 1A or 1B with the tubing 104 including a dual limb breathing circuit and the patient coupling 106 including a non-invasive ventilation mask, according to an example embodiment of the present disclosure. In this embodiment, the tubing 104 of the ventilator system 100 includes a humidifier assembly 302, which is configured to receive air from the mechanical ventilator 102 via humidifier tubing 304. The inspiratory limb 202 connects the humidifier assembly 302 to a temperature probe 306, which is connected to the patient coupling 106. The temperature probe 306 is electrically connected to the humidifier assembly 302 via a heater wire 308. The humidifier assembly 302 is configured to add water vapor to the air before inhalation by the patient. Water is provided to the humidifier assembly 302 from a fluidly coupled water bag 309 or another container. The heater wire 308 may also increase a temperature of exhaled air to a specified temperature using feedback from the temperature probe 306 to minimize ‘rain out’ in the expiratory limb 210.

It should be appreciated that the ventilator system 100 shown in FIGS. 2 and 3 are only examples of possible tubing 104 and patient couplings 106. Any combination of a single limb breathing circuit, a dual limb breathing circuit, or a coax breathing circuit for the tubing 104 may be used in any combination with a nasal cannula, a non-invasive ventilation mask, or an endotracheal tube. Further, any combination of the tubing 104 and the patient couplings 106 may include or omit the humidifier assembly 302 and the associated fluid couplings.

Returning to FIG. 1A, the mechanical ventilator 102 includes or is communicatively coupled to a display screen 108. The example display screen 108 is configured to display one or more physiological parameters and/or ventilator input parameters via one or more graphical user interfaces (“GUIs”). The display screen 108 is also configured to display ventilator modes, ventilator settings, such as tidal volume and inspiratory pressure, and/or one or more alarms. The display screen 108 may include one or more control interfaces, such as buttons or switches for receiving operator inputs. Additionally or alternatively, the display screen 108 includes a capacitive touch screen for receiving operator inputs.

As shown in FIG. 1A, the mechanical ventilator 102 is configured to receive air and/or oxygen from one or more gas sources 110. In some embodiments, the mechanical ventilator 102 is fluidly coupled to an oxygen gas source and an air gas source. Each of the gas sources may be fluidly coupled to a respective port or connector provided on a housing of the mechanical ventilator 102. The one or more gas sources 110 are pressurized to enable the mechanical ventilator 102 to provide air to the patient via the tubing 104 and the patient coupling 106.

In some embodiments, one or more of the gas sources 110 do not include pressurized gas. Instead, the gas sources 110 include a blower or turbine that is integrally formed or included within the mechanical ventilator 102. The blower or turbine is configured to pull ambient air into the mechanical ventilator 102. Internal air flow components, such as air intake vents, filters, valves and/or pressure regulators control the air flow. In some instances, the mechanical ventilators 102 may concentrate oxygen from the ambient air when an oxygen gas source 110 is needed.

To enable air from the one or more gas sources 110 to reach the tubing 104, the mechanical ventilator 102 includes a gas delivery unit 112, one or more flow sensors 114, and one or more pressure sensors 116. The gas delivery unit 112 is configured to use valves and/or pressure regulators to control the pressure, flow rate, and/or gas composition of the gas received from the one or more gas sources 110. The flow sensor(s) 114 is configured to measure a volume of air that is moved through the gas delivery unit 112. The pressure sensor(s) 116 is configured to measure a gas pressure.

The gas delivery unit 112 includes valves 118 for proportioning the mixing of oxygen and air based on selected ventilator settings. In an example, the gas sources 110 are connected to respective connectors of the mechanical ventilator 102. Proportional solenoid valves 118 may be located downstream or upstream from the pressure sensors 116 and/or flow sensors 114 to enable the proportional mixing of the air and oxygen within a mixing manifold and to control the patient volume and patient airway pressure. After mixing, a downstream oxygen sensor (located in an inspiration module of the gas delivery unit 112) measures a concentration of oxygen, which is used to ensure the mixed air has the specified oxygen concentration.

The gas delivery unit 112 may also include an expiratory module and an exhalation control module that is connected to the tubing 104. The expiratory module includes at least one valve 118 and flow sensor 114 for controlling the exhale of air from the patient. The exhalation control module includes one or more proportional solenoid valves 118, regulators (not shown), and pressure sensors 116 to enable at least some of the exhaled air to be recirculated and combined with the air and oxygen from the gas sources 110. In some embodiments, the exhalation control module is omitted such that all exhaled air is discharged from the mechanical ventilator 102.

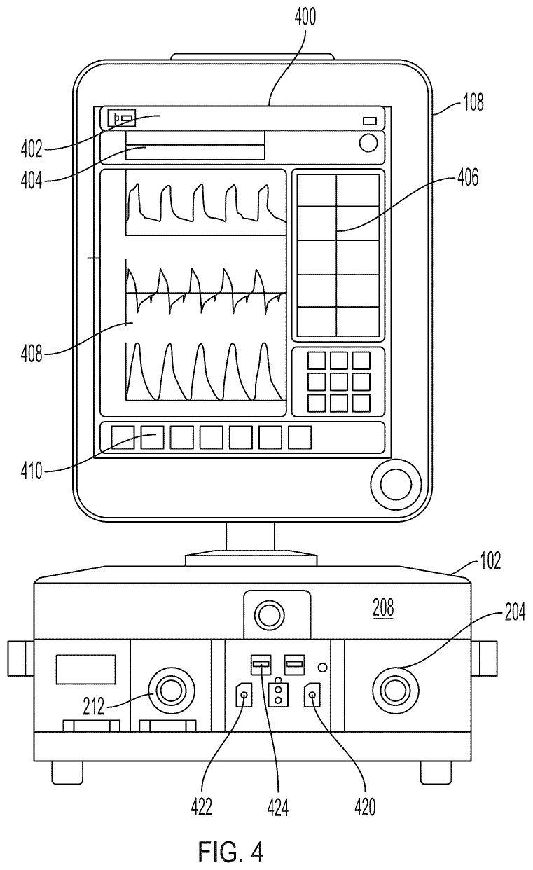

FIG. 4 is a diagram of an example mechanical ventilator 102 of the ventilator system 100 of FIGS. 1A or 1B, according to an example embodiment of the present disclosure. In the illustrated embodiment, the mechanical ventilator 102 is the NKV-550Series produced by Nihon Kohden®. In other embodiments, other models of mechanical ventilators may be used.

The display screen 108 is mechanically and electrically coupled to the housing 208 of the mechanical ventilator 102 to form a single unit. The display screen 108 includes different area or sections to facilitate ventilation therapies, which are shown in one or more graphical user interfaces 400. A status bar area 402 is configured to show a status of the mechanical ventilator 102, patient information such as a name, identifier, gender, age, weight, etc., and battery information. An alarms area 404 is configured to show alarm messages and include a control interface for setting alarms and pausing alarms. A numeric monitor area 406 shows a current value of selected input parameters and/or patient physiological parameters. The monitor area 406 may also display setting values. A waveform area 408 displays a time series graph of selected input parameters and/or patient physiological parameters. A setting area 410 shows control interfaces for selecting a ventilator mode and one or more control settings.

FIG. 4 also shows that the housing 208 of the mechanical ventilator 102 includes one or more ports and interfaces including the inspiratory port 204 and the expiratory port 204. The housing 208 also includes a first interface 420 that is configured to accept a connector for a CO₂ sensor and a second interface 422 that is configured to accept a connector for a SpO₂ sensor. The CO₂ sensor is configured to provide measurements for inspired CO₂ and/or an end tidal CO₂ level (e.g., % or mmHg). The SpO₂ sensor is configured to provide measurements for a percentage of SpO₂ and/or pulse rate specified in beats per minute. The housing 208 further includes one or more communication interfaces 424, such as a universal serial bus (“USB”) interface to enable, for example, an Aerogen nebulizer to be connected. A rear of the housing 208 may include ports for connecting respectively to high pressure air gas sources 110, such as an air source and an oxygen source. The housing 208 may also include one or more Ethernet ports to enable the mechanical ventilator to communicatively couple to a network.

Returning to FIG. 1A, the example mechanical ventilator 102 also includes a processor 120 and a communicatively coupled memory device 122. The processor 120 may include a microcontroller, a microprocessor, a logic controller, an application specific integrated circuit, or combinations thereof. The memory device 122 may include a flash drive, a solid state drive, or a hard disk drive. The memory device 122 stores one or more instructions within one or more control algorithms 124. Execution of the control algorithms 124 by the processor 120 cause the processor 120 to perform the operations described herein.

The example processor 120 is configured to receive signals from the flow sensors 114, the pressure sensors 116, and/or the air sensors. The processor 120 uses the signals to determine when adjustments are needed for the valves 118. For example, signals from the flow sensors 114 are used by the processor 120 to determine whether the air and oxygen are being mixed within a mixing manifold in specified proportions. Further, flow and/or pressure sensors 114 and 116 within the expiratory module of the gas delivery unit 112 transmit signals that are used by the processor 120 to determine volume and flow exhaled from the patient. The data from the flow sensors 114 and the pressure sensors 116 may be used by the processor 120 to estimate the patient respiratory compliance, airway resistance, and respiratory muscle pressure. These parameters may be used by the processor 120 to estimate the patient size and patient respiratory status and facilitate the closed loop control described herein.

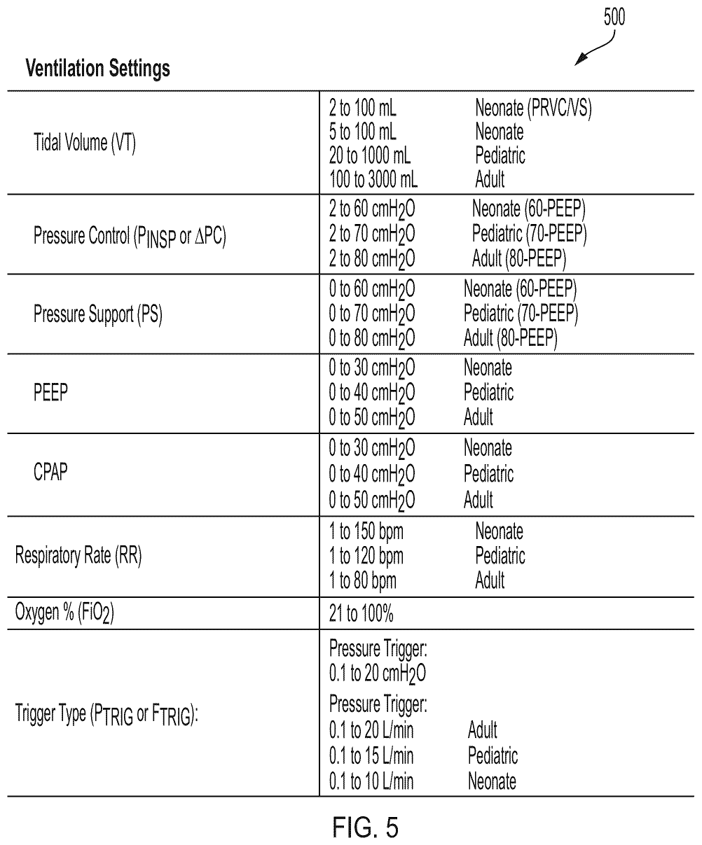

FIG. 5 shows a diagram of example ventilator settings 500 for the mechanical ventilator 102 of FIGS. 1A or 1B, according to an example embodiment of the present disclosure. Each setting 500 includes acceptable ranges based on a patient type. The settings 500 include a tidal volume, a pressure control a pressure support, a PEEP, a CPAP, a respiratory rate, an inspired oxygen level (e.g., F_{I}O₂), and a pressure trigger type. It should be appreciated that the mechanical ventilator 102 may include additional or fewer settings. For example, additional settings may specify a slope of pressurization regarding how quickly a pressure target is to be achieved after a breath is triggered, an expiratory trigger sensitivity specifying at what point a breath cycles to exhalation, and a maximum inspiratory time. The processor 120 may select values for each of the settings 500 based on specified target input parameters, measured input parameters, and/or physiological parameters. The relation between the setting values and the specified target input parameters, measured input parameters, and/or physiological parameters are defined by the control algorithms 124.

It should be appreciated that some of the settings 500 are used only for certain ventilation modes. In the illustrated example, the control algorithms 124 specify at least two ventilation modes including a mandatory breath mode, a spontaneous breath mode, and/or a mixed mode. In other examples, the control algorithms 124 may specify additional or fewer modes. The mandatory breath mode may include an assisted/controlled mechanical ventilation (“A/CMV”) mode, a volume control (“VC”) mode, a pressure control (“PC”) mode, and/or a pressure-regulated volume control (“PRVC”) mode. The mixed mode may include a synchronized intermittent mechanical ventilation (“SIMV”) mode and/or a bilevel positive airway pressure (“BiPAP”) mode. The spontaneous breath mode may include a CPAP mode, a pressure support ventilation (“PSV”) mode, a proportional assist ventilation (“PAV”) mode, and/or a high flow oxygen therapy (“HFOT”) mode. Certain modes may only be available for invasive and non-invasive respiratory treatments.

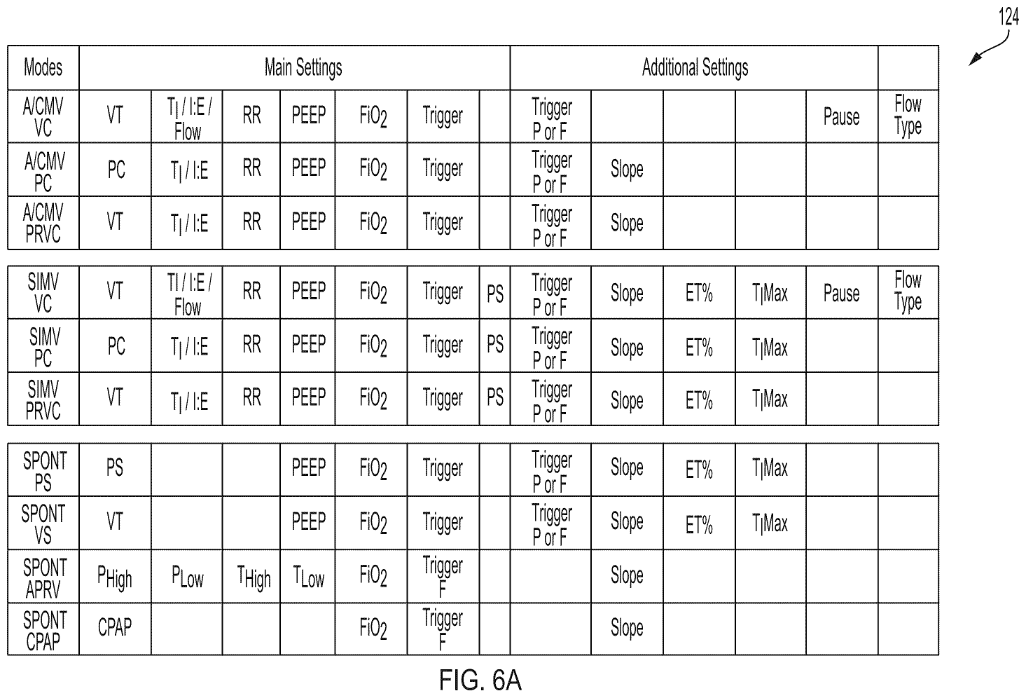

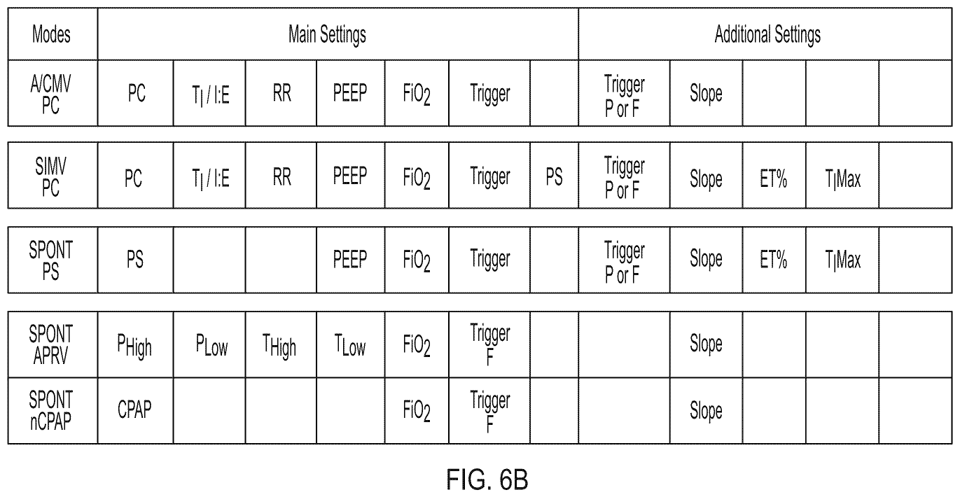

FIGS. 6A and 6B show diagrams of the control algorithms 124 that specify certain ventilator settings 500 based on a corresponding ventilation modes, according to an example embodiment of the present disclosure. Values for each of the modes may be selected by the control algorithm 124 based on a patient age, weight, gender, and/or other physiological parameter values. For the A/CMV mode, a ventilator breath can be triggered by patient or by time, and is terminated by time, where only mandatory breaths are delivered. The respiratory rate setting determines the minimum number of mandatory breaths delivered each minute and the minimum time interval between each breath. These breaths may be time-triggered, patient-triggered, or a combination of both. The inspiratory phase length is determined by the inspiratory time. At the end of the inspiratory phase, the ventilator enters the expiratory phase during which the airway pressure is maintained by the ventilator at the PEEP setting.

In another example, SIMV is a mode in which mandatory breaths and spontaneous breaths coexist. The clinician may choose PC, VC, or PRVC for mandatory breaths, and pressure support (“PS”) to augment spontaneous breaths. The respiratory rate setting determines the total number of mandatory breaths delivered each minute and establishes a timing window that determines whether a patient trigger results in a mandatory breath or a spontaneous breath. The trigger setting (“FTRIG or PTRIG”) determines the airway pressure or patient flow threshold that the patient's effort must reach in order to trigger mandatory breaths as well as to trigger spontaneous breaths in between mandatory breaths. The inspiratory trigger may also be initiated by a patient neural inspiratory signal, such as in Neurally Adjusted Ventilatory Assist (“NAVA”) of the Maquet Servo-U® ventilator. When SIMV is first initiated, a time-triggered mandatory breath is delivered. Thereafter, the first patient-triggered breath in any breath interval is a patient-triggered mandatory breath. Additional breaths within the same interval may be patient-triggered spontaneous breaths.

For spontaneous breaths (“SPONT”), all breaths delivered to the patient by the mechanical ventilator 102 are spontaneous breaths.

For the VC (volume control) mode, the mechanical ventilator 102 is configured to deliver a clinician-set tidal volume over a clinician-set inspiratory time (based on the settings of tidal volume (“VT”), Flow, etc.). VC ventilation provides time-cycled, volume-limited mandatory breaths. Volume and flow (or inspiratory time) are clinician set parameters and the airway pressure is not directly managed by the mechanical ventilator 102. The clinician has the option to choose either square flow type or descending flow type.

For the PC (pressure control) mode, the mechanical ventilator 102 is configured to deliver a clinician-set inspiratory pressure target (“P_{INSP}”) or a change in PC (“ΔPC”) over a clinician-set inspiratory time. P_{INSP} sets the target pressure above zero, while ΔPC sets the target value above PEEP. A Slope % setting determines how fast the airway pressure reaches the set pressure level after the breath is initiated. Higher Slope % settings result in more rapid increases in inspiratory pressure. The inspiratory phase of a pressure control breath terminates when the set inspiratory time has elapsed.

For the PRVC mode, the mechanical ventilator 102 is configured to deliver pressure control breaths with a variable inspiratory pressure target to achieve a clinician-set target tidal volume. The inspiratory pressure target is adjusted on a breath-by-breath basis by the ventilator 102 to achieve the targeted tidal volume. The Slope % setting determines how fast the airway pressure reaches the target pressure level after the breath is initiated.

For the PSV mode, the mechanical ventilator 102 is configured to trigger patient breaths using FTRIG or PTRIG. Once triggered, the ventilator delivers a clinician-set inspiratory pressure above PEEP. The Slope % setting determines how fast the airway pressure reaches the set pressure level after the breath is triggered. A higher Slope % setting results in more rapid increases in inspiratory pressure. The pressure support breath can be terminated by one of three predetermined termination criteria including flow, time, and pressure, whichever threshold is hit first. When the inspiratory flow decreases to a set expiratory trigger (“ET %”) of inspiratory peak flow, flow termination criterion will end the pressure support inspiration. Increasing the ET % setting results in a shorter pressure support inspiratory time and decreasing the ET % setting results in a longer pressure support inspiratory time. When the elapsed inspiratory time has reached to the clinician set maximum pressure support time (i.e., T_{Max} PS), time termination criterion ends the pressure support inspiration.

For volume support (“VS”) modes, the mechanical ventilator 102 is configured to manage the pressure support level breath-by-breath to achieve a set target tidal volume. The Slope % setting determines how fast the airway pressure reaches the target pressure level after the breath is initiated.

The control algorithms 124 are accordingly configured to specify how certain ventilator settings are to be adjusted to maintain certain set parameters, as provided above. The specified input parameter may be manually entered into the mechanical ventilator 102 via the display screen 108 or transmitted to the mechanical ventilator 102 over a network as an electronic respiratory prescription. The control algorithms 124 may also specify how certain ventilator settings and/or modes are to be adjusted based on measured input parameters and/or physiological parameters.

As shown in FIGS. 1A or 1B, the mechanical ventilator 102 includes one or more physiological sensors 130. To measure neurological physiological parameters indicative of patient awareness, the sensor 130 includes a bispectral index (“BIS”) monitor that measures electroencephalogram (“EEG”) signals of a patient. Alternatively, the sensor 130 includes an EEG sensor that transmits EEG signals to the processor 120, which uses the EEG signals to determine the BIS value. The sensor 130 may also include a train-of-four (“TOF”) monitor that outputs a neuromuscular value as a neurological parameter value. The sensor 130 may further include an intracranial pressure (“ICP”) sensor configured to measure an ICP, which is output as an ICP neurological parameter value.

The physiological sensor 130 may additionally or alternatively include a monitor or sensor to measure a metabolic parameter value. To measure a patient body temperature, the sensor 130 includes a temperature sensor. To measure a heart rate parameters, the sensor 130 includes a heart rate monitor or an electrocardiogram monitor. In addition, the sensor 130 may further include a blood pressure sensor, a mean arterial pressure sensor, a blood oxygen sensor, a weight scale, etc. In some embodiments, the processor 120 is configured to determine the metabolic parameter value from a patient diagnosis that is received from at least one the graphical user interface 400 of the display screen 108, a respiratory treatment setting or target input parameter, and/or an EMR. The patient diagnosis includes at least one of a disease, a health condition, a laboratory result, or specified medical procedures.

The physiological sensor 130 may additionally or alternatively include a monitor or sensor to measure a circulatory parameter value that is indicative of at least one of a cardiac output, an arterial blood pressure, a central venous blood pressure, or a pulse rate. The sensor 130 may include a blood pressure monitor, a heart rate monitor, and/or a patient bedside monitor. In some embodiments, the processor 120 is configured to determine the circulatory parameter value from a patient diagnosis that is received from at least one of the graphical user interface 400 of the display screen 108, a respiratory treatment setting or target input parameter, and/or an EMR.

As discussed in more detail below, the processor 120 of FIGS. 1A or 1B is configured to receive physiological parameter values from one or more physiological sensors 130. The processor 120 applies the received physiological parameter values to the one or more control algorithms stored in the memory device 122. Based on a result of the comparison, the processor 120 is configured to maintain a current operating mode and/or settings of the mechanical ventilator 102, change at least one mode of the mechanical ventilator 102, change at least one setting of the mechanical ventilator 102, or change at least one setting and at least one mode of the mechanical ventilator 102. In some embodiments, some settings and/or modes 500 may be associated with ranges of physiological parameter values and/or groups of different types of physiological parameters. For instance, one mode may be selected based on a combination of circulatory parameters and/or metabolic parameters.

FIG. 1B shows a diagram of the ventilator system 100 including the mechanical ventilator 102 and a separate computing device 140, according to an example embodiment of the present disclosure. In this illustrated example, the separate computing device 140 includes the processor 120 and the memory device 122 with the control algorithm(s) 124 discussed above in connection with FIG. 1A for providing closed loop control using physiological parameter values. The computing device 140 may include a Raspberry Pi™ computer, a smartphone, a tablet computer, a laptop computer, or other stand-alone computing device. The computing device 140 is communicatively coupled to a processor 120a of the mechanical ventilator 102 via a wired connection (e.g., a USB connection, a Lightning™ connection, a high-definition multimedia interface (“HDMI”) connection, an Ethernet connection, a serial connection, etc.) or a wireless connection (e.g., a Bluetooth® connection, a Wi-Fi connection, a Zigbee® connection, etc.).

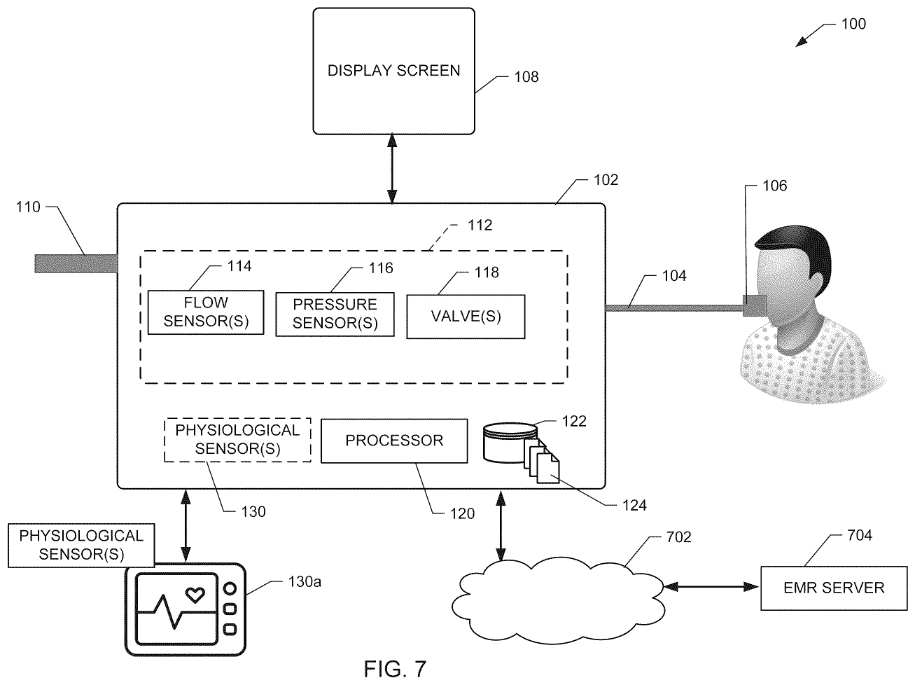

In the illustrated example, the processor 120 of the computing device 140 is configured to create and/or adjust an electronic respiratory prescription for the mechanical ventilator 102 using physiological parameter values. Additionally or alternatively, the processor 120 is configured to set or adjust one or more ventilator settings 500 and/or ventilator modes using received physiological parameter values, as discussed herein. In some embodiments, the computing device 140 may include one or more physiological sensors 130a and/or be communicatively coupled to one or more physiological sensors 130a via a wired or wireless connection. Additionally or alternatively, the computing device 140 may be connected to a network 702 and/or an EMR server 704, as shown and discussed in connection with FIGS. 7 to 9.

The computing device 140 is configured to receive physiological parameter values from external or local physiological sensors 130, 130a. The computing device 140 applies the one or more control algorithms 124 to the physiological parameter values, as disclosed herein, to determine one or more ventilator setting adjustments, mode adjustments, and/or an electronic respiratory prescription. The computing device 140 transmits the electronic respiratory prescription, selected settings, and/or selected modes to the processor 120a of the mechanical ventilator 102. The processor 120a is configured to operator the flow sensor(s) 114, the pressure sensor(s) 116, and/or the valve(s) 118 according to the electronic respiratory prescription, the selected settings, and/or the selected modes. In some embodiments, the processor 120a is configured to determine settings and/or modes based on ventilator input parameter values or target input parameter values that are specified within the electronic respiratory prescription provided by the processor 120 of the computing device 140.

In this embodiment, the processor 120a of the mechanical ventilator 102 is configured to control a patient's breathing. The processor 120 of the separate computing device 140 is configured to determine how the patient's breathing is adjusted based on physiological parameter values. Such a configuration enables a separate device to be used to update the operations of, for example, known mechanical ventilators 102. For instance, the computing device 140 may be connected to the communication interface 424 to enable a known mechanical ventilator to provide closed loop control using the physiological parameter values.

FIG. 7 shows another diagram of the ventilator system 100 of FIGS. 1A or 1B, according to an example embodiment of the present disclosure. In this embodiment, a physiological sensor(s) 130a is external to and directly communicatively coupled to the mechanical ventilator 102. Additionally, the mechanical ventilator 102 may include one or more physiological sensors 130, as discussed above in conjunction with FIG. 1. However, in some embodiments, the physiological sensor 130 is omitted. The physiological sensor(s) 130a may be communicatively coupled to the processor 120 (e.g., a transceiver of the processor 120) of the mechanical ventilator 102 via at least one of a USB connection, a micro-USB connection, an HDMI connection, a Bluetooth® connection, a Zigbee® connection, a Wi-Fi connection, etc. In some instances, the physiological sensor(s) 130a are connected to the processor 120 via a mesh or direct Internet-of-Things (“IoT”) protocol.

In some embodiments, the physiological sensor(s) 130a may be integrally formed or connected to a patient monitor, which is communicatively coupled to the mechanical ventilator 102. Together, the patient monitor and the sensors constitute the physiological sensor(s) 130a disclosed herein. The patient monitor may include a bedside computer, a tablet computer, a laptop computer, etc. that is configured to display physiological parameters. The sensors are hardwired or wirelessly connected to the monitor, which is communicatively coupled to the processor 120 of the mechanical ventilator 102. The patient monitor may be configured to convert signals from the sensors into formatted digital data, which is transmitted to the processor 120 as physiological parameter values.

FIG. 7 also shows that the processor 120 of the mechanical ventilator 102 is communicatively coupled to a network 702 (e.g., a wide area network, local area network, a wireless local area network, an Ethernet, the Internet, a cellular network, or combinations thereof). In the illustrated example, the processor 120 communicates with at least an EMR system 704 via the network 702. The EMR system 704 includes, for example, a server and a database of patient EMR records stored on a memory device. In some embodiments, the EMR system 704 may include a cloud-based database. The processor 120 transmits a request message to the EMR system 704 to obtain, for example, an EMR of a patient. The request message includes an identifier of the patient, which enables the EMR system 704 to identify one or more corresponding patient EMRs, which are then transmitted to the processor 120.

An EMR may include physiological parameter values including a patient diagnosis, laboratory results, prescribed medications, physiological parameter values (e.g., weight, blood pressure, body temperature, heart rate, etc.), patient physiological values (e.g., height, gender, age, etc.), and/or a patient's medical history. The processor 120 is configured to use keyword searching and/or metadata of known fields to extract one or more physiological parameter values for processing through one or more of the control algorithms 124. In other instances, the request message identifies physiological parameter types such that the EMR server 704 only returns relevant data without the processor 120 having to search for the physiological parameter values.

In some instances, the EMR server 704 may receive the physiological parameter values from one or more physiological sensors that have monitored or are monitoring the patient. In this manner, the physiological parameter values are stored in the EMR system 704 before being transmitted to the mechanical ventilator. As one can appreciate, the mechanical ventilator 102 can receive physiological parameter values from virtually any device to enable the processor 120 to determine when ventilator modes and/or settings are to be adjusted.

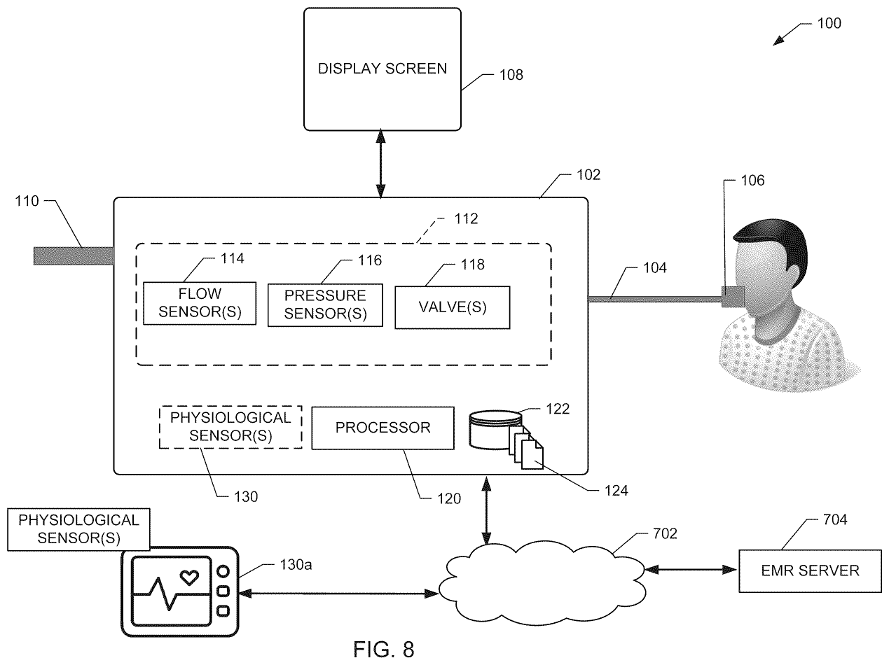

FIG. 8 shows a further diagram of the ventilator system 100 of FIG. 7, according to an example embodiment of the present disclosure. In this embodiment, the physiological sensor(s) 130a are communicatively coupled to the network 702 instead of directly connected to the processor 120 of the mechanical ventilator 102. The physiological sensor 130a, either the patient monitor or the sensor itself, may be connected to the network 702 via a Wi-Fi connection, a cellular connection, an Ethernet connection, or combinations thereof.

In some instances, the physiological sensor(s) 130a is configured to transmit physiological parameters values to the EMR server 704. The physiological sensor(s) 130a may transmit the physiological parameters values in one or more messages that include a patient identifier. The EMR server 704 uses the patient identifier to search for and store the physiological parameters values to a corresponding patient EMR. The EMR server 704 then transmits (or transmits upon request) the physiological parameters values to the processor 120 of the mechanical ventilator 102.

In other instances, the physiological sensor 130 is configured to transmit the physiological parameters values to the processor 120 via the network 702. The physiological sensor 130 may communicate with the processor 120 using known Internet Protocol addresses, media access control addresses, or other identifier of the mechanical ventilator 102. In some embodiments, the physiological parameters values are encrypted or otherwise transmitted using a secure communication channel. The use of the network 702 enables any Internet-enabled physiological sensor 130a to transmit physiological parameters values to the processor 120.

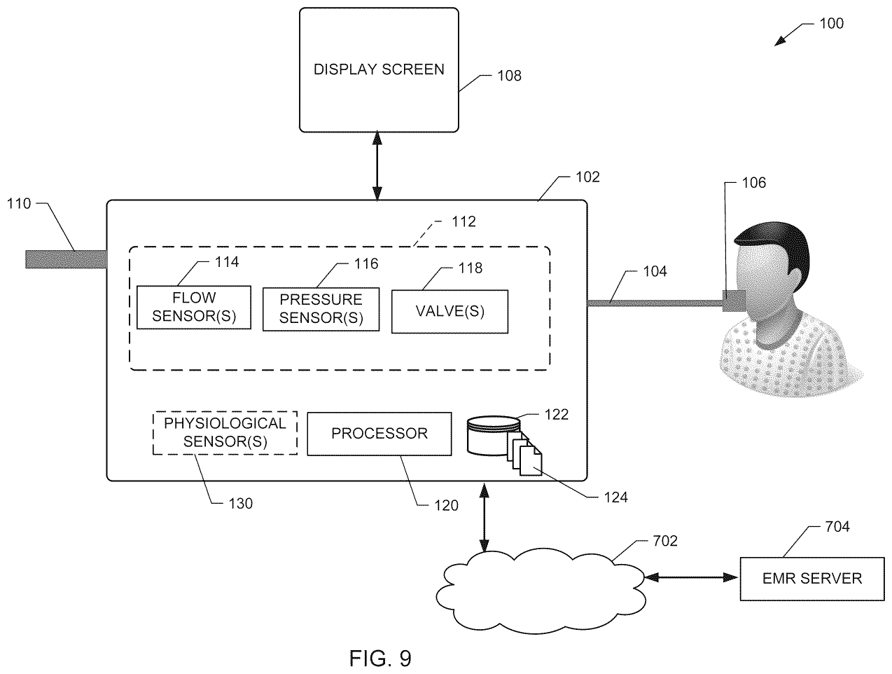

FIG. 9 shows another diagram of the ventilator system 100 of FIG. 7, according to an example embodiment of the present disclosure. In this example, the external physiological sensor(s) 130a are omitted. Instead, the processor 120 of the mechanical ventilator 102 is configured to receive physiological parameters values from the EMR server 704 and/or local physiological sensors 130.

In the examples of FIGS. 7 to 9, the processor 120 of the mechanical ventilator 102 is communicatively coupled to the EMR server 704. In addition to receiving physiological parameters values, the processor 120 may also receive an electronic respiratory prescription from the EMR server 704. The electronic respiratory prescription may be created at a clinician computer and stored to the EMR server 704. The electronic respiratory prescription is then transmitted from the EMR server 704 to the mechanical ventilator 102.

The electronic respiratory prescription may specify one or more ventilator modes, settings, and/or target input parameters (e.g., respiratory treatment settings) for the mechanical ventilator 102. The processor 120 selects settings 500 and/or modes using the control algorithms 124 discussed above based on the electronic respiratory prescription. The processor 120 subsequently causes a respiratory treatment to be administered by the mechanical ventilator according to the electronic respiratory prescription.

### Closed Loop Ventilator Control Embodiments

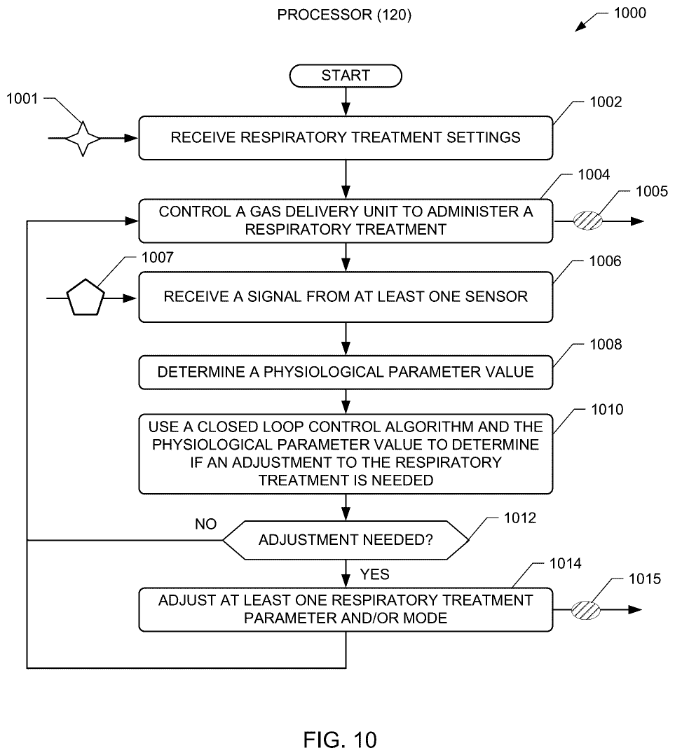

FIG. 10 shows a flow diagram illustrating an example procedure 1000 for close loop control of the mechanical ventilator 102 of FIGS. 1 to 9, according to an example embodiment of the present disclosure. The example procedure 1000 may be carried out by, for example, the processor 120, the gas delivery unit 112, the pressure regulators, the flow and pressure sensors 114 and 116, the valves 118, the physiological sensor(s) 130, and/or the physiological sensor(s) 130a described in conjunction with FIGS. 1 to 9. In an example, the processor 120 may execute one or more of the control algorithms 124 to perform the operations described herein. Although the procedure 1000 is described with reference to the flow diagram illustrated in FIG. 10, it should be appreciated that many other methods of performing the functions associated with the procedure 1000 may be used. For example, the order of many of the blocks may be changed, certain blocks may be combined with other blocks, and many of the blocks described are optional.

The procedure 1000 begins when the processor 120 of the mechanical ventilator 102 receives one or more respiratory treatment settings 1001 (block 1002). The respiratory treatment settings 1001 may be input into a control interface of the display screen 108. Additionally or alternatively, the respiratory treatment settings 1001 may be included within an electronic respiratory prescription that is received from in the processor 120 from the EMR server 704 via the network 702. The processor 120 uses the respiratory treatment settings 1001 to control the gas delivery unit 112, the flow and pressure sensors 114 and 116, and/or the valves 118 of the mechanical ventilator 102 to administer a respiratory treatment (block 1004). In some instances, the processor 120 uses the control algorithms 124 to select values for settings 500 and select a ventilator mode for the mechanical ventilator 102 based on the respiratory treatment settings 1001. To enable the respiratory treatment to be provided, the processor 120 transmits one or more control instructions or signals 1005 to the gas delivery unit 112, and/or the valves 118 based on the settings 500 and/or ventilation mode, as discussed in conjunction with FIGS. 5 and 6. For instance, the processor 120 may select the A/CMV mode and then select values for tidal volume, pressure control, pressure support, PEEP, respiratory rate, inspired oxygen, inspiratory time, and an inspiratory trigger function.

After the treatment has started, the processor 120 receives respiratory input parameter values from the flow and pressure sensors 114 and 116 in addition to signals from CO₂ and SpO₂. The processor 120 uses the control algorithms 124 to determine whether the settings 500 are to be modified based on the received input parameter values. The processor 120 continuously or periodically performs this check as additional respiratory input parameter values are received during the treatment.

In addition to providing closed loop control based on respiratory input parameter values, the processor 120 additionally or alternatively provides closed loop control using physiological parameter values. The processor 120 receives physiological parameter values from at least one physiological sensor 130 and 130a (block 1006). The physiological parameter values may be included in one or more messages 1007 and/or transmitted via one or more analog signals. From the messages 1007 and/or signals, the processor 120 determines the physiological parameter values (block 1008).

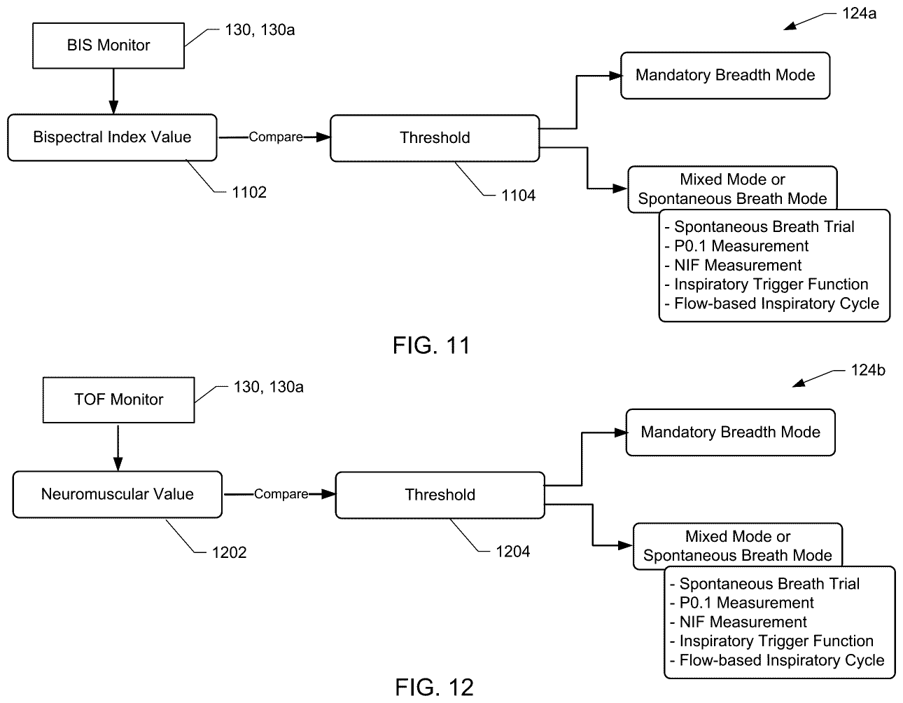

The processor 120 then uses one or more of the closed loop control algorithms 124 in conjunction with the determined physiological parameter values (and optionally previously received physiological parameter values) to determine if an adjustment to the respiratory treatment is needed (block 1010). The control algorithms 124 may specify, for example, conditions for changing one or more settings 500 and/or ventilation modes based on certain values of one or more types of physiological parameters. FIGS. 11 to 14 provide examples of control algorithms 124.

When no adjustment is needed (block 1012), the procedure 1000 returns to controlling the gas delivery unit 112, the pressure regulators, and/or the valves 118 of the mechanical ventilator 102 to administer a respiratory treatment (block 1004). When an adjustment is needed (block 1012), the processor 120 applies the adjustment to one or more settings 500 and/or changes a mode of the mechanical ventilator 102 (block 1014). The processor 120 may adjust the setting and/or mode by transmitting control instructions or signals 1015 to the gas delivery unit 112, the pressure regulators, and/or the valves 118. As such, the processor 120 adjusts the respiratory treatment based on a current physiological condition of the patient. This ensures the patient receives a respiratory treatment that is appropriate for their current state, thereby improving patient outcomes and treatment efficiency. The processor 120 then continues controlling the gas delivery unit 112, the pressure regulators, and/or the valves 118 of the mechanical ventilator 102 and makes subsequent adjustments based on later received respiratory input parameter values and/or physiological parameter values. The procedure 1000 continues until the respiratory treatment ends.

### Mechanical Ventilator Control Algorithm Embodiments

As disclosed above, the processor 120 of the mechanical ventilator 102 is configured to use one or more control algorithms 124 to determine when one or more respiratory treatment settings 1001 and/or modes are to be adjusted during a treatment. FIGS. 11 to 15 show diagrams that are illustrative of at least some control algorithms 124a to 124e, according to example embodiments of the present disclosure. It should be appreciated that the control algorithms 124 may include additional conditions or adjust additional (or fewer) settings. Further, some control algorithms 124 may use a plurality of different types of physiological parameters to determine how certain settings and/or modes are to be selected.

The control algorithm 124a of FIG. 11 is configured to use a BIS value 1102 (e.g., a physiological (neurological) parameter value) that is received from a BIS monitor physiological sensor 130a (or 130). In other instances, the processor 120 receives an EEG signal from an EEG physiological sensor 130a. In these other instances, the processor 120 determines the BIS value 1102 from the EEG signal. As shown, the control algorithm 124a compares the BIS value 1102 to a threshold 1104, which may include a BIS value between 60 to 70. Alternatively, the threshold 1104 may be entered by a clinician via the graphical user interface 400 of the display screen 108 and/or included within an electronic respiratory prescription or specified by respiratory treatment settings. In some instances, the threshold is based on patient type (e.g., neonate, pediatric, adult, etc.).

Based on the comparison with the threshold 1104, the control algorithm 124 either selects a mandatory breath mode, a spontaneous breath mode, or a mixed mode. In the illustrated example, the control algorithm 124a specifies that the mandatory breath mode is to be selected when the BIS value 1102 falls below a threshold 1104. In addition to switching to the mandatory breath mode, the control algorithm 124a may also specify that a spontaneous breath trial should not be performed, a P0.1 measurement should not be performed, and/or an inspiratory trigger function should be disabled.

When the BIS value 1102 is above the threshold 1104, the control algorithm 124a enables the mixed mode or the spontaneous breath mode to be used for the respiratory treatment. When the mechanical ventilator 102 is already in one of these modes, the control algorithm 124a causes the processor 120 to remain in the programmed mode. When the mechanical ventilator 102 is in the mandatory breath mode and the BIS value 1102 exceeds the threshold 1104, the control algorithm 124a either selects the mixed mode or the spontaneous breath mode or prompts a clinician to select one of the modes. In addition to switching to the mixed mode or the spontaneous breath mode, the control algorithm 124a may also specify that a spontaneous breath trial should be performed, a P0.1 measurement should be performed, a negative inspiratory force NIF measurement should be performed, and/or an inspiratory trigger function should be enabled.

The processor 120 of the mechanical ventilator 102 uses the control algorithm 124a to accordingly detect when a patient has decreased brain activity. After detecting that a patient has decreased brain activity, the control algorithm 124a specifies that a mandatory breath mode and corresponding settings are used for the respiratory treatment. This ensures that the mechanical ventilator 102 provides more forceful breathing for a patient when a patient is, for example, unconscious.

As discussed above, the mandatory breath mode includes at least one of an assisted/controlled mechanical ventilation (“A/CMV”) mode, a volume control (“VC”) mode, a pressure control (“PC”) mode, a pressure regulated volume control (“PRVC”) mode, and a mode analog to one of these modes. Additionally, the spontaneous breath mode includes at least one of a continuous positive airway pressure (“CAPC”) mode, a pressure support ventilation (“PSV”) mode, a volume support ventilation (“VS”) mode, a proportional assist ventilation (“PAV”) mode, a high flow oxygen therapy (“HFOT”) mode, and a mode analog to one of these modes. Further, the mixed mode includes at least one of a synchronized intermittent mechanical ventilation (“SIMV”) mode, a bilevel positive airway pressure (“BiPAP”) mode, and a mode analog to one of these modes. The ventilator modes disclosed herein are not all-inclusive. Those skilled in the art should appreciate that other ventilator modes that still belong to either a mandatory mode, a mixed mode, or a spontaneous mode may be used. For example, a NAVA (Neurally Adjusted Ventilatory Assist) mode may be used in the closed loop control described herein as a mixed mode or a spontaneous mode.

FIG. 12 shows a control algorithm 124b that uses a neuromuscular value 1202 as a physiological (neurological) parameter value, according to an example embodiment of the present disclosure. In this example, the physiological sensor 130a includes a TOF monitor, which outputs the neuromuscular value 1202. Alternatively, the TOF monitor transmits a signal, which is processed by the processor 120 to determine a corresponding neuromuscular value. The TOF monitor may be used to assess a neuromuscular block within a patient. The neuromuscular value 1202 may be indicative, for example, of muscular paralysis and is used by the processor 120 to determine when a patient needs more assistance breathing.

The control algorithm 124b of FIG. 12 specifies that when the neuromuscular value 1202 is below a threshold 1204 (indicative of muscular paralysis), the processor 120 is to switch to the mandatory breath mode. Additionally, the control algorithm 124b may specify that when the neuromuscular value 1202 is below the threshold 1204, the processor 120 is configured to refrain from performing at least one of a spontaneous breath trial, a P0.1 measurement, a negative inspiratory force NIF measurement, and/or an inspiratory trigger function.

The control algorithm 124b of FIG. 12 also specifies that when the neuromuscular value 1202 is above the threshold 1204 (indicative of patient muscular activity), the processor 120 is to switch to the mixed mode or the spontaneous breath mode. In some embodiments, the processor 120 uses other physiological parameter values to select between the mixed mode and the spontaneous breath mode. Alternatively, the processor 120 may prompt an operator via the display screen 108 to select between the two different modes. In yet alternative embodiments, the processor 120 is configured to revert back to which of the modes was originally programmed or specified in an electronic respiratory prescription. In addition to above, the control algorithm 124b may specify that when the neuromuscular value 1202 is above the threshold 1204, the processor 120 is configured to perform at least one of a spontaneous breath trial, a P0.1 measurement, a NIF measurement, and/or an inspiratory trigger function.

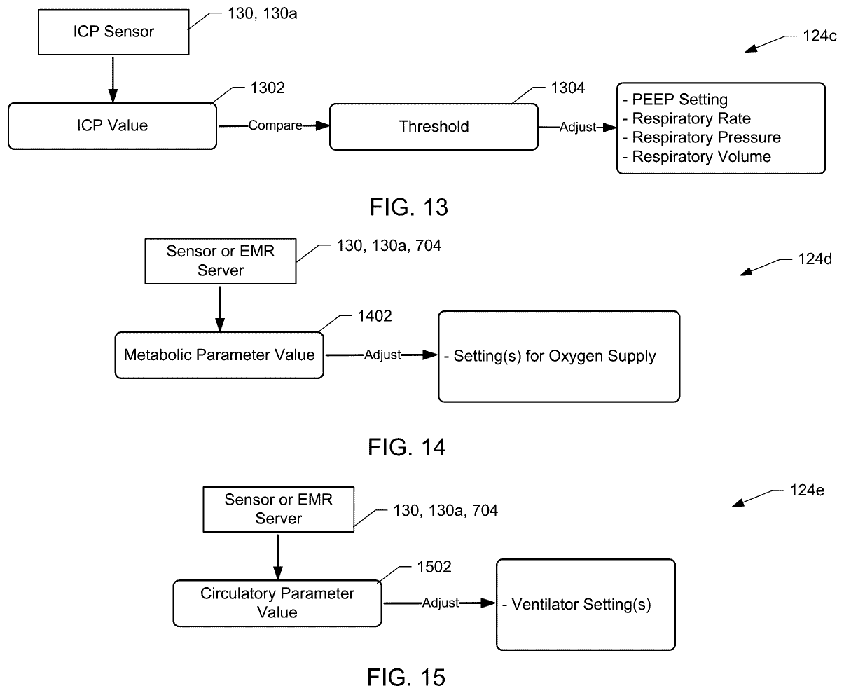

FIG. 13 shows a control algorithm 124c that uses an intracranial pressure (“ICP”) value 1302 as a physiological (neurological) parameter value. The mechanical ventilator 102 may include an ICP physiological monitor 130. Alternatively, the IPC monitor 130a may be communicatively coupled to the processor 120 of the mechanical ventilator 102, as discussed above.

The illustrated control algorithm 124c is configured to relate PEEP settings to changes to ICP values 1302. The control algorithm 124c is also configured to relate minute ventilation-related settings to ICP values 1302. During a treatment, the mechanical ventilator 102 is configured to operate according to respiratory treatment settings 1001 specifying a PEEP setting and minute ventilation-related settings. The example minute ventilation-related settings include at least one of a respiratory rate, a respiratory pressure, or a respiratory volume.

During the treatment, the processor 120 is configured to receive the ICP value 1302 from the ICP monitor 130a. The processor 120 uses the control algorithm 124c to compare the ICP value 1302 to a threshold 1304, which may be 15 mmHg. As shown in FIG. 13, the control algorithm 124c specifies that when the ICP value 1302 is greater than the threshold 1304, the processor 120 adjusts the PEEP setting based on a reaction of the ICP value 1302 to the previous PEEP setting (and/or previous adjustment made to a PEEP setting). Additionally, the processor 120 is configured to adjust at least one of the minute ventilation-related settings 500 to maintain the ICP value 1302. The control algorithm 124c also specifies that when a certain direction or magnitude of the previous PEEP setting adjustment results in an increase in the ICP value 1302, the processor 120 is configured to avoid or minimize changing the PEEP setting to that certain direction or magnitude to avoid deterioration of the patient's ICP. Such a configuration accordingly ensures that the mechanical ventilator 102 proactively maintains a health (or acceptable) ICP for a patient under treatment.

In some embodiments, the control algorithm 124c also takes into account end tidal CO₂ values, which may be received via the first interface 420 from a CO₂ sensor. In these embodiments, the processor 120 is configured to determine actual minute ventilation according to the end tidal CO₂ values. The control algorithm 124c specifies that the processor 120 is to adjust the minute ventilation-related settings such that the actual minute ventilation is maintained high enough to prevent ICP values from increasing.

In addition to neurological parameter values, the processor 120 may use control algorithms 124 that relate ventilator modes and/or settings 500 to metabolic physiological parameters. FIG. 14 shows a control algorithm 124d that uses a metabolic parameter value 1402 as a physiological parameter value. Patient metabolic parameters may include, for example, patient body temperature (including surface temperature and/or core temperature) and/or patient diagnosis. As a patient's body temperature or other metabolic functions increase, the patient generally needs greater amounts of oxygen. The control algorithm 124d accordingly automatically adjusts the mechanical ventilator 102 to ensure a patient receives sufficient oxygen to match their current metabolic physiological condition.

In the illustrated example, the control algorithm 124d relates ventilator settings 500 for patient oxygen supply to metabolic parameter values 1402. The settings for oxygen supply may include a respiratory rate, an inspiratory pressure, an inspiratory volume, or an inspiratory oxygen concentration. For a respiratory treatment, the processor 120 receives respiratory treatment settings for a patient, where at least one of the respiratory treatment settings specifies a patient oxygen supply setting. During the treatment, the processor 120 receives the metabolic parameter values 1402 from a sensor 130 of the mechanical ventilator 102 and/or a communicatively coupled sensor 130a. Additionally or alternatively, the metabolic parameter value 1402 is indicative of a patient diagnosis (or temperature) that is received from at least one of the graphical user interface 400 of the mechanical ventilator 102, a patient EMR from the EMR server 704, or included within the respiratory treatment settings. The patient diagnosis includes, for example, at least one of a disease, a health condition, a laboratory result, or specified medical procedures.

The processor 120 is configured to use the control algorithm 124d to determine whether at least one of the ventilator settings 500 for patient oxygen supply is to be adjusted based on the metabolic parameter values 1402. In an example, the control algorithm 124d may specify that for every increase in the metabolic parameter value 124d, minute oxygen delivery specified by the settings for the patient oxygen supply is to be increased by a specified percentage. In some instances, the specified percentage is between 8% and 15%. In other instances, the specified percentage is at least one of a percentage between 8% and 15%, received from the graphical user interface 400 of the mechanical ventilator 102, or included within the respiratory treatment settings.

In some embodiments, the control algorithm 124d also takes into account F_{I}O₂ values, which may be received via the interface 422 from a F_{I}O₂ sensor. In these embodiments, the processor 120 is configured to calculate oxygen delivery as a patient minute ventilation multiplied by an average F_{I}O₂ value. The patient minute ventilation is a sum of a patient inspiratory volume of each breath over a one minute duration or an average patient inspiratory volume of each breath multiplied by a respiratory rate over a one minute duration. The control algorithm 124d specifies how one or more oxygen settings are accordingly adjusted based on current or planned oxygen delivery.

FIG. 15 shows a control algorithm 124e that uses a circulatory parameter value 1502 as a physiological parameter value. In this embodiment, the control algorithm 124e relates ventilator settings to circulatory parameter values. The processor 120 is configured to receive the circulatory parameter values 1502 from one or more sensors 130, 130a and use the control algorithm 124e to adjust one or more ventilator settings and/or operating modes.

In some embodiments, the circulatory parameter value 1502 is indicative of at least one of a cardiac output, an arterial blood pressure, a central venous blood pressure, or a pulse rate. In these embodiments, the circulatory parameter value 1502 is received from a sensor 130, 130a that includes at least one of a blood pressure monitor, a heart rate monitor, or a patient bedside monitor. Alternatively, the processor 120 receives the circulatory parameter value 1502 from the EMR server 704.

In the illustrated example, the control algorithm 124e specifies that a PEEP setting is to be reduced, maintained, or increased gradually when at least one of (i) cardiac output is low, (ii) the central venous blood pressure is high, and/or (iii) the arterial blood pressure is low. The control algorithm 124e may also specify that the PEEP setting is to be reduced, maintained, or increased gradually when an increase in a PEEP setting results in at least one of (i) an unproportioned cardiac output reduction, (ii) a central venous blood pressure increase, or (iii) an arterial blood pressure decrease. Further the control algorithm 124e may specify adjusting the PEEP setting by adjusting a respiratory rate and/or respiratory volume. The control algorithm 124e adjusts a patient's breathing to help maintain or adjust their cardiac function to acceptable levels.

In some embodiments, the control algorithm 124e is configured to indicate (or the processor 120 is configured to determine) that a patient's circulatory function is compromised based on at least one of the cardiac output, the arterial blood pressure, the central venous blood pressure, or the pulse rate. In response, the control algorithm 124e specifies that the processor 120 is to adjust the PEEP ventilator setting after determining the patient's circulatory function is compromised. Additionally or alternatively, the control algorithm 124e specifies that the processor 120 is to adjust a F_{I}O₂ ventilator setting after determining the patient's circulatory function is compromised. Moreover additionally or alternatively, the control algorithm 124e specifies that the processor 120 is to adjust a F_{I}O₂ ventilator setting after determining the patient's circulatory function has improved.

It should be appreciated that the control algorithms 124a to 124e shown respectively in FIGS. 11 to 15 are only representative of the plurality of control algorithms 124 that may be used by the processor 120 of the mechanical ventilator 102. Further, the processor 120 may use multiple control algorithms 124 at the same time so that the ventilator 102 is adjusted in different manners based on all the physiological parameter values received. In some instances, the processor 120 may use a control algorithm that specifies which settings and/or modes are not compatible such that results from multiple control algorithms 124 do not conflict with each other.

These control algorithms may provide a hierarchy of importance regarding physiological parameter values to ensure the mechanical ventilator 102 does not negatively affect a patient's health. For instance, circulatory and/or respiratory physiological parameters may be given precedence over metabolic and neurological.

### Conclusion

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should also be appreciated that 35 U.S.C. 112(f) or pre-AIA 35 U.S.C. 112, paragraph 6 is not intended to be invoked unless the terms “means” or “step” are explicitly recited in the claims. Accordingly, the claims are not meant to be limited to the corresponding structure, material, or actions described in the specification or equivalents thereof.

1. A mechanical ventilator system comprising:
a sensor configured to measure a neurological parameter;
a memory device storing a closed loop control algorithm that relates neurological parameter values to ventilation modes including a mandatory breath mode, a spontaneous breath mode, and a mixed mode;
a mechanical ventilator including a gas delivery unit configured to control a flow of gas from at least one gas source to a patient; and
a processor communicatively coupled to the sensor, the memory device, and the gas delivery unit, the processor configured to:
receive respiratory treatment settings for a patient, at least one of the respiratory treatment settings specifying one of the ventilation modes as a programmed ventilation mode,
control the gas delivery unit to administer a respiratory treatment according to the treatment settings including the programmed ventilation mode,
receive a signal from the sensor,
determine a neurological parameter value from the signal,
use the closed loop control algorithm and the neurological parameter value to determine the programmed ventilation mode is to be adjusted to another one of the ventilation modes as an adjusted ventilation mode, and
control the gas delivery unit to administer the respiratory treatment according to the adjusted ventilation mode.

2. The system of claim 1, wherein (i) the sensor includes electroencephalogram (“EEG”) sensors and the processor is configured to determine, from the signal, a bispectral index (“BIS”) value as the neurological parameter value, or (ii) the sensor includes a BIS monitor that provides the BIS value based on measured EEG signals,
wherein the adjusted ventilation mode is the mandatory breath mode, and
wherein the processor is configured to switch to the mandatory breath mode when the BIS value falls below a threshold.

3. The system of claim 2, wherein the threshold is at least one of:
a BIS value between 60 to 70;
received via a user interface of the mechanical ventilator; or
included within the respiratory treatment settings.

4. The system of claim 2, wherein the processor is further configured to, after switching to the mandatory breath mode, refrain from performing at least one of:
a spontaneous breath trial,
a P0.1 measurement using a pressure sensor of the mechanical ventilator, or
an inspiratory trigger function.

5. The system of claim 2, wherein the adjusted ventilation mode is the mixed mode or the spontaneous breath mode, and
wherein the processor is configured to switch to the mixed mode or the spontaneous breath mode when the BIS value rises above the threshold.

6. The system of claim 5, wherein the processor is further configured to, after switching to the mixed mode or the spontaneous breath mode, perform at least one of:
a spontaneous breath trial,
a P0.1 measurement using a pressure sensor of the mechanical ventilator,
a negative inspiratory force (“NIF”) measurement, or
an inspiratory trigger function with a sensitivity so sensitive that the processor attempts to induce a patient spontaneous trigger effort.

7. The system of claim 1, wherein
the mandatory breath mode includes at least one of an assisted/controlled mechanical ventilation (“A/CMV”) mode, a volume control (“VC”) mode, a pressure control (“PC”) mode, a pressure regulated volume control (“PRVC”) mode, and a mode analog to one of these modes,
the spontaneous breath mode includes at least one of a continuous positive airway pressure (“CAPC”) mode, a pressure support ventilation (“PSV”) mode, a volume support ventilation (“VS”) mode, a proportional assist ventilation (“PAV”) mode, a high flow oxygen therapy (“HFOT”) mode, and a mode analog to one of these modes, and
the mixed mode includes at least one of a synchronized intermittent mechanical ventilation (“SIMV”) mode, a bilevel positive airway pressure (“BiPAP”) mode, and a mode analog to one of these modes.

8. The system of claim 1, wherein the sensor is remote from the mechanical ventilator and communicatively coupled to the processor via at least one of a directed wired connection, a wireless connection, or a network connection.

9. The system of claim 1, wherein the sensor includes a train-of-four (“TOF”) monitor and the processor is configured to determine, from the signal, a neuromuscular value as the neurological parameter value,
wherein the adjusted ventilation mode is the mandatory breath mode, and
wherein the processor is configured to switch to the mandatory breath mode when the neuromuscular value is indicative of muscular paralysis.

10. : The system of claim 9, wherein the processor is further configured to, after switching to the mandatory breath mode, refrain from performing at least one of:
a spontaneous breath trial,
a P0.1 measurement using a pressure sensor of the mechanical ventilator,
a negative inspiratory force (“NIF”) measurement, or
an inspiratory trigger function.

11. The system of claim 9, wherein the adjusted ventilation mode is the mixed mode or the spontaneous breath mode, and
wherein the processor is configured to switch to the mixed mode or the spontaneous breath mode when the neuromuscular value is indicative of patient muscular activity.

12. The system of claim 11, wherein the processor is further configured to, after switching to the mixed mode or the spontaneous breath mode, perform at least one of:
a spontaneous breath trial,
a P0.1 measurement using a pressure sensor of the mechanical ventilator,
a negative inspiratory force (“NIF”) measurement, or
an inspiratory trigger function.

13. The system of claim 1, wherein the gas delivery unit includes a gas inlet fluidly coupled to the at least one gas source, a pump and/or valve, a flow sensor, and a humidifier.

14. The system of claim 1, wherein the processor and the memory device are one of:
separate from the mechanical ventilator; or
included within the mechanical ventilator.

15. A mechanical ventilator system comprising:
an intracranial pressure (“ICP”) sensor configured to measure an ICP;
a memory device storing a closed loop control algorithm that relates positive end expiratory pressure (“PEEP”) settings to changes to ICP values and relates minute ventilation-related settings to ICP values;
a mechanical ventilator including a gas delivery unit configured to control a flow of gas from at least one gas source to a patient; and
a processor communicatively coupled to the sensor, the memory device, and the gas delivery unit, the processor configured to:
receive respiratory treatment settings for a patient, at least one of the respiratory treatment settings specifying a PEEP setting and minute ventilation-related settings,
control the gas delivery unit to administer a respiratory treatment according to the treatment settings,
receive an ICP value from the ICP sensor,
compare the ICP value to a threshold,
when the ICP value is greater than the threshold, adjust the PEEP setting based on a reaction of the ICP value to a previous PEEP setting adjustment, and
adjust at least one of the minute ventilation-related settings to maintain the ICP value.

16. The system of claim 15, wherein when a certain direction or magnitude of the previous PEEP setting adjustment results in an increase in the ICP value, the processor is configured to avoid or minimize changing the PEEP setting to that certain direction or magnitude to avoid deterioration of the ICP.

17. The system of claim 15, wherein the mechanical ventilator further includes a sensor configured to measure end tidal CO₂ values, and
wherein the processor is further configured to:
determine actual minute ventilation according to the end tidal CO₂ values, and
adjust the minute ventilation-related settings such that the actual minute ventilation is maintained high enough to prevent ICP values from increasing.

18. The system of claim 15, wherein the minute ventilation-related settings include at least one of a respiratory rate, a respiratory pressure, or a respiratory volume.

19. The system of claim 15, wherein the threshold is 15 mmHg.

20. The system of claim 15, wherein the processor and the memory device are one of:
separate from the mechanical ventilator; or
included within the mechanical ventilator.

21. A mechanical ventilator system comprising:
a memory device storing a closed loop control algorithm that relates ventilator settings for patient oxygen supply to metabolic parameter values;
a gas delivery unit of a mechanical ventilator configured to control a flow of gas from at least one gas source to a patient; and
a processor communicatively coupled to the sensor, the memory device, and the gas delivery unit, the processor configured to:
receive respiratory treatment settings for a patient, at least one of the respiratory treatment settings specifying a patient oxygen supply setting,
control the gas delivery unit to administer a respiratory treatment according to the treatment settings,
receive a metabolic parameter value, and
adjust at least one of the ventilator settings for patient oxygen supply based on the metabolic parameter value.

22. The system of claim 21, wherein the metabolic parameter value is indicative of a patient body temperature.

23. The system of claim 22, wherein the control loop algorithm specifies that for every increase in the metabolic parameter value, minute oxygen delivery specified by the settings for the patient oxygen supply is to be increased by a specified percentage.

24. The system of claim 23, wherein the specified percentage is between 8% and 15%.

25. The system of claim 23, wherein the specified percentage is at least one of:
a percentage between 8% and 15%;
received via a user interface of the mechanical ventilator; or
included within the respiratory treatment settings.

26. The system of claim 21, wherein the metabolic parameter value is indicative of a patient diagnosis that is received from at least one of:
a user interface of the mechanical ventilator apparatus;
an electronic medical record (“EMR”) server via a network; or
included within the respiratory treatment settings.

27. The system of claim 26, wherein the patient diagnosis includes at least one of a disease, a health condition, a laboratory result, or specified medical procedures.

28. The system of claim 21, wherein the settings for oxygen supply include a respiratory rate, an inspiratory pressure, an inspiratory volume, or an inspiratory oxygen concentration.

29. The system of claim 28, wherein the processor is further configured to calculate oxygen delivery as a patient minute ventilation multiplied by an average fraction of inspired oxygen (“F_{I}O₂”) value,
wherein the patient minute ventilation is a sum of a patient inspiratory volume of each breath over a one minute duration or an average patient inspiratory volume of each breath multiplied by a respiratory rate over a one minute duration.

30. The system of claim 21, wherein the processor and the memory device are one of:
separate from the mechanical ventilator; or
included within the mechanical ventilator.

31. A mechanical ventilator system comprising:
a memory device storing a closed loop control algorithm that relates ventilator settings to circulatory parameter values;
a gas delivery unit of a mechanical ventilator configured to control a flow of gas from at least one gas source to a patient; and
a processor communicatively coupled to the sensor, the memory device, and the gas delivery unit, the processor configured to:
receive ventilator settings for a patient,
control the gas delivery unit to administer a respiratory treatment according to the ventilator settings,
receive a circulatory parameter value, and
adjust at least one of the ventilator settings based on the circulatory parameter value.

32. The system of claim 31, wherein the circulatory parameter value is indicative of at least one of a cardiac output, an arterial blood pressure, a central venous blood pressure, or a pulse rate, and
wherein the circulatory parameter value is received from at least one of a blood pressure monitor, a heart rate monitor, a patient bedside monitor, or an electronic medical record (“EMR”) server.

33. The system of claim 32, wherein the control algorithm specifies that a positive end expiratory pressure (“PEEP”) ventilator setting is to be reduced, maintained, or increased gradually when at least one of (i) cardiac output is low, (ii) the central venous blood pressure is high, and/or (iii) the arterial blood pressure is low.

34. The system of claim 32, wherein the control algorithm specifies that a positive end expiratory pressure (“PEEP”) ventilator setting is to be reduced, maintained, or increased gradually when an increase in a PEEP setting results in at least one of (i) an unproportioned cardiac output reduction, (ii) a central venous blood pressure increase, or (iii) an arterial blood pressure decrease.

35. The system of claim 34, wherein the control algorithm specifies adjusting the PEEP ventilator setting by adjusting a respiratory rate and/or respiratory volume.

36. The system of claim 32, wherein the processor is further configured to:
determine that a patient's circulatory function is compromised based on at least one of the cardiac output, the arterial blood pressure, the central venous blood pressure, or the pulse rate; and
use the control algorithm to adjust a positive end expiratory pressure (“PEEP”) ventilator setting after determining the patient's circulatory function is compromised.

37. The system of claim 32, wherein the processor is further configured to:
determine that a patient's circulatory function is compromised based on at least one of the cardiac output, the arterial blood pressure, the central venous blood pressure, or the pulse rate; and
use the control algorithm to adjust a fraction of inspired oxygen (“F_{I}O₂”) ventilator setting after determining the patient's circulatory function is compromised.

38. The apparatus of claim 37, wherein the processor is further configured to:
determine that the patient's circulatory function is improving based on at least one of the cardiac output, the arterial blood pressure, the central venous blood pressure, or the pulse rate; and
use the control algorithm to adjust the fraction of inspired oxygen (“F_{I}O₂”) ventilator setting after determining the patient's circulatory function has improved.

39. The system of claim 31, wherein the processor and the memory device are one of:
separate from the mechanical ventilator; or
included within the mechanical ventilator.
